(19) United States
(12) Patent Application Publication
(10) Pub. No.: US 2026/0144962 A1
(43) Pub. Date: May 28, 2026

(54) Medical Device Monitoring System and Method

(71) Applicant: Bard Access Systems, Inc., Salt Lake City, UT (US)

(72) Inventors: Steffan Sowards, Salt Lake City, UT (US); Stefan Josef Fellner, Salt Lake City, UT (US); Amelia Marie Smith, Rochester, MI (US)

(21) Appl. No.: 18/961,405

(22) Filed: Nov. 26, 2024

(51) Int. Cl. A61M 25/01

(52) U.S. Cl. CPC A61M 25/0102

(57) ABSTRACT

A catheter placement system for placing a catheter, the system includes a stylet with an optical fiber extending therethrough, the stylet body extending between a proximal end and a distal tip. A console, equipped with optical logic, delivers broadband incidence light to the optical fiber and receives reflected light signals to determine positional information about the stylet body. The stylet body is configured to be separable at a point between the proximal end and distal tip to allow a catheter to be advanced over a distal portion of the stylet body. The distal portion is configured to be reattached to the proximal portion to reconfirm the position of the stylet body distal tip prior to removal of the stylet from the catheter. The system further includes a severing device that is easy to actuate and ensures a clean, perpendicular cut to facilitate reconnection between the proximal and distal portions.

### BACKGROUND

Current approaches for catheter placement systems typically involve the use of stylets or guidewires to advance the catheter to a target location within the vasculature. These conventional systems lack the ability to provide real-time positional information about the stylet or guidewire during the catheter placement procedure, leading to potential inaccuracies and inefficiencies in the process. To resolve these, various tracking systems have been developed to track the location of the catheter assembly within the vasculature to improve placement accuracy. These systems often employ a variety of modalities. Fluoroscopic tracking modalities expose the patient to potentially harmful radiation. Electromagnetic (“EM”) tracking modalities can be susceptible to interference from nearby sources of EM radiation. Acoustic tracking modalities require skilled personnel to operate and may be limited in image clarity.

Fiber-optic shape sensing (“FOSS”) systems have been developed to overcome some of these limitations. FOSS tracking systems provide a clear image of the tracked catheter, are less susceptible to magnetic or electromagnetic interference, and do not expose to patient to harmful radiation. However, the optical fibers used in existing catheter placement systems can be fragile and are limited in their ability to flex without rupturing. Further, existing optical fibers may not be easily separable and reconnectable, limiting potential placement methods as well as limiting the ability to switch out damaged or ruptured optical fibers during a procedure.

Disclosed herein are embodiments directed to fiber enabled placement systems configured to be disconnected and optionally reconnected to facilitate placement of a catheter and to address the foregoing.

### SUMMARY

In some aspects, the techniques described herein relate to a catheter placement system for placing a catheter within a vasculature of a patient including, a stylet having a body extending along a longitudinal axis between a console connector at a proximal end and a distal tip, the stylet body including an optical fiber extending therethrough, the stylet body severable along an axis extending perpendicular to the longitudinal axis to separate a distal portion of the body from a proximal portion of the body and configured to be reconnected to communicatively couple the optical fiber of the distal portion with the optical fiber of the proximal portion, and a console having an optical logic and providing broadband incidence light to the optical fiber and configured to receive reflected light signals from the optical fiber, the optical logic configured to determine positional information about the stylet body.

In some aspects, the techniques described herein relate to a catheter placement system accordingly to claim 1, wherein the catheter is configured to slidably engage the severed proximal end of the stylet body distal portion to advance a catheter distal tip to a target location within the vasculature of the patient.

In some aspects, the techniques described herein relate to a catheter placement system accordingly to claim 1, further including a severing device having a body defining a channel, a portion of the stylet body extending through the channel and an actuator including a cutting edge configured to cut the stylet body perpendicular to the longitudinal axis of the stylet body.

In some aspects, the techniques described herein relate to a catheter placement system accordingly to claim 1, wherein the stylet body includes a keying feature extending along a longitudinal length of the body and configured to orient the stylet distal portion about the longitudinal axis relative to the stylet proximal portion.

In some aspects, the techniques described herein relate to a catheter placement system accordingly to claim 4, further including a connector having a body defining a lumen and including an aligning feature extending along an inner surface of the connector lumen, the aligning feature configured to engage a first keying feature of the stylet distal portion and a second keying feature of the stylet proximal portion to align an optical fiber end of the stylet distal portion with an optical fiber end of the stylet proximal portion.

In some aspects, the techniques described herein relate to a catheter placement system accordingly to claim 5, wherein the connector engages one or both of the stylet distal portion and the stylet proximal portion in one of a friction fit engagement, a press fit engagement, a snap fit engagement, a threaded engagement, a bayonet engagement, a latching engagement, an adhesive, a bonding, a welding, an ultrasonic welding, or an RF energy welding.

In some aspects, the techniques described herein relate to a catheter placement system accordingly to claim 3, wherein the channel of the severing device includes a fiduciary marker having a predetermined non-linear path to a portion of the channel.

In some aspects, the techniques described herein relate to a catheter placement system accordingly to claim 1, wherein the stylet body includes a plurality of magnetic elements extending longitudinally through the stylet body and configured to releasably attach a proximal end of the stylet distal portion to a distal end of a stylet proximal portion.

In some aspects, the techniques described herein relate to a method of placing a catheter including, advancing a distal end of a body of a stylet through an insertion site to a target location, sending broadband incident light from a console to an optical fiber of the stylet body, receiving reflected light to the console to determine positional information of the stylet body, confirming a distal end of the stylet body is at the target location, severing a distal portion of the stylet body from a proximal portion of the stylet body, advancing a catheter over the stylet distal portion subsequent to severing the distal portion of the stylet body, and removing the stylet distal portion from a lumen of the catheter.

In some aspects, the techniques described herein relate to a method, further including coupling the proximal end of the distal portion of the stylet body with a distal end of the proximal portion of the stylet body subsequent to the step of advancing the catheter over the stylet distal portion and receiving reflected light from the distal portion to determine positional information of the distal portion.

In some aspects, the techniques described herein relate to a method accordingly to claim 10, wherein coupling the proximal end of the distal portion of the stylet body with the distal end of the proximal portion of the stylet body includes aligning a keying feature of the stylet body with an alignment feature of a connector, the keying feature extending along a longitudinal length of the body.

In some aspects, the techniques described herein relate to a method accordingly to claim 11, wherein the connector has a connector body defining a lumen, the alignment feature extending longitudinally along an inner surface of the lumen to align an optical fiber end of the stylet distal portion with an optical fiber end of the stylet proximal portion.

In some aspects, the techniques described herein relate to a method accordingly to claim 12, wherein the connector engages one or both of the stylet distal portion and the stylet proximal portion in one of a friction fit engagement, a press fit engagement, a snap fit engagement, a threaded engagement, a bayonet engagement, a latching engagement, an adhesive, a bonding, a welding, an ultrasonic welding, or an RF energy welding.

In some aspects, the techniques described herein relate to a method, wherein severing the distal portion from the proximal portion further includes actuating a severing device having a body defining a channel, a portion of the stylet body extending through the channel and an actuator including a cutting edge transitionable along an axis perpendicular to a longitudinal axis of the stylet body.

In some aspects, the techniques described herein relate to a method accordingly to claim 14, wherein the channel of the severing device includes a fiduciary marker having a predetermined non-linear path to a portion of the channel.

In some aspects, the techniques described herein relate to a method accordingly to claim 9, wherein the stylet body includes a plurality of magnetic elements extending longitudinally through the stylet body and configured to releasably attach a proximal end of the stylet distal portion to a distal end of a stylet proximal portion.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 shows a schematic view of a fiber optic enabled medical device monitoring system, in accordance with embodiments disclosed herein.

FIG. 2 shows a perspective view of a stylet assembly for use with the system of FIG. 1, in accordance with embodiments disclosed herein.

FIG. 3 shows a perspective view of a stylet and catheter assembly for use with the system of FIG. 1, in accordance with embodiments disclosed herein.

FIG. 4 shows the system of FIGS. 1-3 inserted into a vasculature of a patient, in accordance with embodiments disclosed herein.

FIG. 5 shows an exemplary structure of a section of the multi-core optical fiber included within the stylet assembly of FIGS. 1-3, in accordance with embodiments disclosed herein.

FIG. 6A shows a perspective view of a multi-core optical fiber included within an elongate medical device, in accordance with embodiments disclosed herein.

FIG. 6B shows a cross-section view of the optical fiber of FIG. 6A, in accordance with embodiments disclosed herein.

FIGS. 7A-7B show a longitudinal cross-sectional view of a severing device and a portion of a stylet body extending therethrough, in accordance with embodiments disclosed herein.

FIG. 7C shows a plan view of a severing device and a portion of a stylet body extending therethrough, in accordance with embodiments disclosed herein.

FIGS. 8A-8C show an exemplary method of use of the system of FIG. 1-3 including a severing device, in accordance with embodiments disclosed herein.

FIG. 9 shows a side view of a connector for coupling a distal portion with a proximal portion of the stylet body, in accordance with embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein. It is understood that the drawings are diagrammatic and schematic representations of exemplary embodiments of the invention, and are neither limiting nor necessarily drawn to scale.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise. Also, the words “including,” “has,” and “having,” as used herein, including the claims, shall have the same meaning as the word “comprising.”

In the following description, the terms “or” and “and/or” as used herein are to be interpreted as inclusive or meaning any one or any combination. As an example, “A, B or C” or “A, B and/or C” mean “any of the following, A, B, C, A and B, A and C, B and C, A, B and C.” An exception to this definition will occur only when a combination of elements, components, functions, steps or acts are in some way inherently mutually exclusive.

The term “logic” is representative of hardware and/or software that is configured to perform one or more functions. As hardware, logic may include circuitry having data processing and/or storage functionality. Examples of such circuitry may include, but are not limited or restricted to a processor, a programmable gate array, a microcontroller, an application specific integrated circuit, combinatorial circuitry, or the like. Alternatively, or in combination with the hardware circuitry described above, the logic may be software in the form of one or more software modules, which may be configured to operate as its counterpart circuitry. The software modules may include, for example, an executable application, a daemon application, an application programming interface (API), a subroutine, a function, a procedure, a routine, source code, or even one or more instructions. The software module(s) may be stored in any type of a suitable non-transitory storage medium, such as a programmable circuit, a semiconductor memory, non-persistent storage such as volatile memory (e.g., any type of random access memory “RAM”), persistent storage such as non-volatile memory (e.g., read-only memory “ROM”, power-backed RAM, flash memory, phase-change memory, etc.), a solid-state drive, hard disk drive, an optical disc drive, or a portable memory device.

With respect to “proximal,” a “proximal portion” or a “proximal end portion” of, for example, a stylet or system disclosed herein includes a portion of the stylet or system intended to be near a clinician when the stylet or system is used on a patient. Likewise, a “proximal length” of, for example, the stylet or system includes a length of the stylet or system intended to be near the clinician when the stylet or system is used on the patient. A “proximal end” of, for example, the stylet or system includes an end of the stylet or system intended to be near the clinician when the stylet or system is used on the patient. The proximal portion, the proximal end portion, or the proximal length of the stylet or system can include the proximal end of the stylet or system; however, the proximal portion, the proximal end portion, or the proximal length of the stylet or system need not include the proximal end of the stylet or system. That is, unless context suggests otherwise, the proximal portion, the proximal end portion, or the proximal length of the stylet or system is not a terminal portion or terminal length of the stylet or system.

With respect to “distal,” a “distal portion” or a “distal end portion” of, for example, a stylet or system disclosed herein includes a portion of the stylet or system intended to be near or in a patient when the stylet or system is used on the patient. Likewise, a “distal length” of, for example, the stylet or system includes a length of the stylet or system intended to be near or in the patient when the stylet or system is used on the patient. A “distal end” of, for example, the stylet or system includes an end of the stylet or system intended to be near or in the patient when the stylet or system is used on the patient. The distal portion, the distal end portion, or the distal length of the stylet or system can include the distal end of the stylet or system; however, the distal portion, the distal end portion, or the distal length of the stylet or system need not include the distal end of the stylet or system. That is, unless context suggests otherwise, the distal portion, the distal end portion, or the distal length of the stylet or system is not a terminal portion or terminal length of the stylet or system.

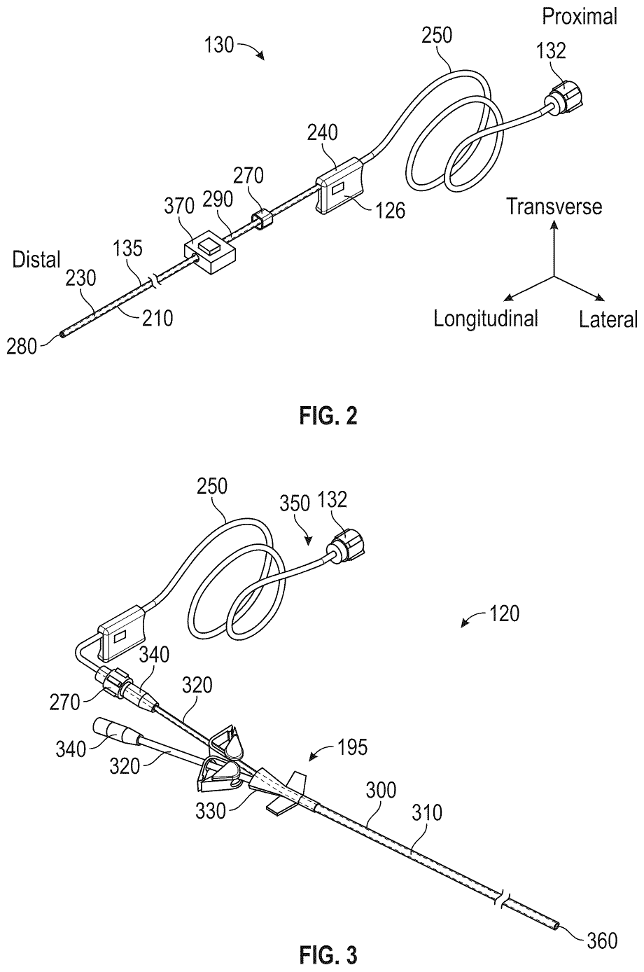

To assist in the description of embodiments described herein, as shown in FIG. 2, a longitudinal axis extends substantially parallel to an axial length of the stylet. A lateral axis extends normal to the longitudinal axis, and a transverse axis extends normal to both the longitudinal and lateral axes.

As used herein, the term “location” is used to indicate a location of the medical device, or portion thereof, in three-dimensional space. By way of example, a distal tip of a medical device can be located at a location defined by three-dimensional co-ordinate. As used herein, the term “orientation” is used to indicate an orientation of the medical device at its location. By way of example, a longitudinal axis of a distal tip of the medical device can be oriented to be aligned along a particular direction in three-dimensional space at a specific location. As used herein, the term “shape” is used to indicate a plain shape of the medical device. By way of example, a ‘J’ shape of a medical device can include an elongate medical device with a curved tip. As used herein, the term “position” combines one or more aspects of the location, orientation and/or shape of a medical device. By way of example, at least a distal portion of the medical device can be in malpositioned when the distal portion of the medical device is folded over itself such that a distal tip of the medical device is oriented away from a desired orientation.

Unless defined otherwise, all technical and scientific terms used herein have the same meaning as commonly understood by those of ordinary skill in the art.

It is important to note that, though the below discussion focuses on usage of a stylet for the placement of a catheter into the body of the patient, the stylet described herein can be employed to place a variety of medical devices, especially other elongate medical devices, in a variety of locations within the patient body. As such, the principles of the present disclosure should not be considered limiting to what is explicitly described herein. Examples of catheter assemblies and medical devices that may benefit from the disclosure may include a peripherally inserted central catheter (“PICC”), central venous catheter (“CVC”), rapidly insertable central catheters (“RICC”), urinary catheter, midline catheter, peripheral catheter, or the like.

In light of the above, a multi-core optical fiber can also be paired with one or more conductive medium for electrical signal monitoring thus serves multiple modalities. For example, the first modality constitutes an optical modality with shape sensing functionality to determine the physical state of the stylet, or similar elongate medical device. The physical state of the stylet provides information to assist a clinician in guiding a catheter assembly to a desired location within the vasculature.

The one or more second modalities can include but not limited to a tip location/navigation system (“TLS”) modality and/or an ECG modality. In an embodiment, a tip location/navigation system (“TLS”) modality includes where the stylet with conductive medium is advanced to detect and avoid any tip malposition during such advancement. In an embodiment, an ECG modality includes wherein ECG signal-based catheter tip guidance is employed to enable tracking and guidance of the stylet/catheter tip to a target location with respect to a node of the patient's heart from which the ECG signals originate.

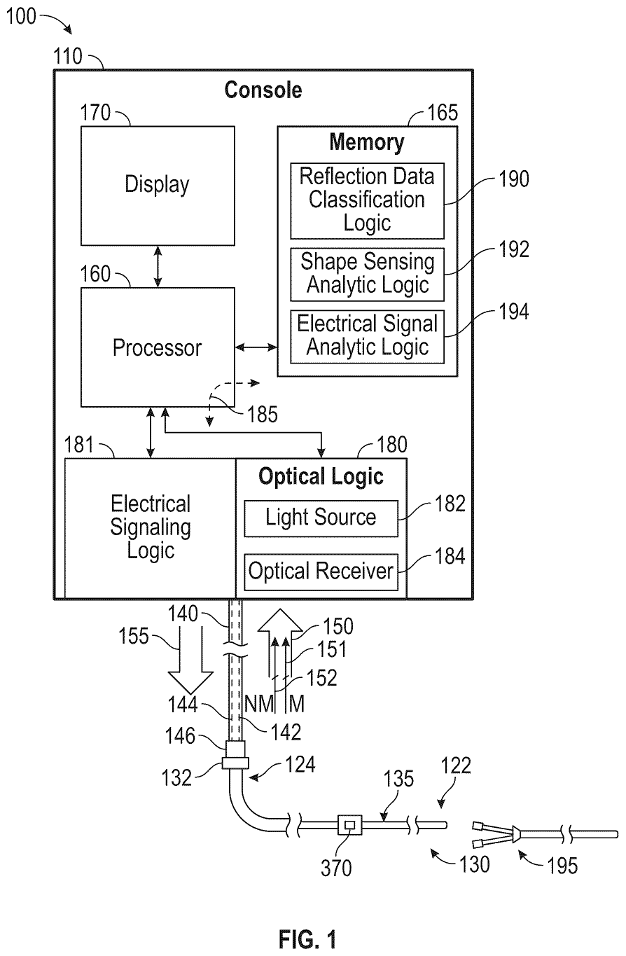

Referring to FIG. 1, an illustrative embodiment of a medical device monitoring system (“system”) 100 is shown. As shown, the system 100 generally includes a console 110 having an interrogator, and a handheld, elongate medical device such as a stylet/catheter assembly 120, 130 communicatively coupled to the console 110. It will be appreciated, however, that the stylet/catheter assembly 120 is exemplary and the assembly 120 can include one or more various stylets, trocars, guidewires, catheters, or combinations thereof. For this embodiment, the stylet/catheter assembly 120 includes a stylet assembly 130, as described in more detail herein.

In an embodiment, the stylet assembly 130 includes a stylet body 290 on its distal end 122 and a console connector 132 on its proximal end 124. The console connector 132 enables the stylet assembly 130 to be operably connected to the console 110 via an interconnect 140 including one or more optical fibers 142 (hereinafter, “optical fiber(s)”) and, optionally, a conductive medium 144 terminated by one or more optical/electric connectors (“connector”) 146. Herein, the connector 146 is configured to engage (mate) with the console connector 132 to allow for the propagation of light between the console 110 and the stylet assembly 130 as well as the propagation of electrical signals from the stylet body 290 to the console 110.

An exemplary implementation of the console 110 includes one or more components, such as a processor 160, a memory 165, a display 170 and one or more logic engines such as an optical logic 180, an electrical signaling logic 181, a reflection data classification logic 190, a shape sensing analytic logic 192, and an electrical signal analytic logic 194. As will be appreciated, one or more of these components can be included in the console 110, or provided as a separate standalone structure and communicatively coupled, wired or wirelessly, with the console 110, or combinations thereof. Although it is appreciated that the console 110 can take one of a variety of forms and may include additional components (e.g., power supplies, ports, interfaces, etc.) that are not directed to aspects of the disclosure. An illustrative example of the console 110 is illustrated in U.S. Pat. No. 10,992,078, the entire contents of which are incorporated by reference herein. The processor 160, with access to the memory 165 (e.g., non-volatile memory), is included to control functionality of the console 110 during operation. As shown, the display 170 may be a liquid crystal diode (LCD) display integrated into the console 110 and employed as a user interface to display information to the clinician, especially during a catheter placement procedure (e.g., cardiac catheterization). In another embodiment, the display 170 may be separate from the console 110. Although not shown, a user interface is configured to provide user control of the console 110. These and other combinations and configurations of the console 110 and associated components are contemplated to fall within the scope of the present invention.

For both of these embodiments, the content depicted by the display 170 may change according to which mode the stylet body 290 is configured to operate, e.g. optical, TLS, ECG, or other modality. In TLS mode, the content rendered by the display 170 may constitute a two-dimensional (2-D) or three-dimensional (3-D) representation of the physical state (e.g., length, shape, form, and/or orientation) of the stylet body 290 computed from characteristics of reflected light signals 150 returned to the console 110. The reflected light signals 150 constitute light of a specific spectral width of broadband incident light 155 reflected back to the console 110. According to one embodiment of the disclosure, the reflected light signals 150 may pertain to various discrete portions (e.g., specific spectral widths) of broadband incident light 155 transmitted from and sourced by the optical logic 180, as described below.

According to one embodiment of the disclosure, an activation control 126, included on the stylet assembly 130, may be used to set the stylet body 290 into a desired operating mode and selectively alter operability the display 170 by the clinician to assist in medical device placement. For example, based on the modality of the stylet body 290, the display 170 of the console 110 can be employed for optical modality-based guidance during catheter advancement through the vasculature or TLS modality to determine the physical state (e.g., length, form, shape, orientation, etc.) of the stylet body 290. In one embodiment, information from multiple modes, such as optical, TLS, ECG, etc., may be displayed concurrently (e.g., at least partially overlapping in time). In one embodiment, the display 170 is a liquid crystal diode (LCD) device or a touch screen device.

Referring still to FIG. 1, the optical logic 180 is configured to support operability of the stylet assembly 130 and enable the return of information to the console 110, which may be used to determine the physical state associated with the stylet body 290 along with monitored electrical signals, such as ECG signaling, electrophysiological sensors, etc., via an electrical signaling logic 181 that supports receipt and processing of the received electrical signals from the stylet body 290 (e.g., ports, analog-to-digital conversion logic, etc.). The physical state of the stylet body 290 may be based on changes in characteristics of the reflected light signals 150 received from the stylet body 290. The characteristics may include shifts in wavelength caused by strain on certain regions of the core fibers integrated within a multi-core optical fiber 135 positioned within or operating as the stylet body 290, as shown below. From information associated with the reflected light signals 150, the console 110 may determine (through computation or extrapolation of the wavelength shifts) the physical state of the stylet body 290.

According to one embodiment of the disclosure, as shown in FIG. 1, the optical logic 180 may include a light source 182 and an optical receiver 184. The light source 182 is configured to transmit the broadband incident light 155 for propagation over the optical fiber(s) 142 included in the interconnect 140, which are optically connected to the multi-core optical fiber 135 within the stylet body 290. In one embodiment, the light source 182 is a tunable swept laser, although other suitable light source can also be employed in addition to a laser, including semi-coherent light sources, LED light sources, etc.

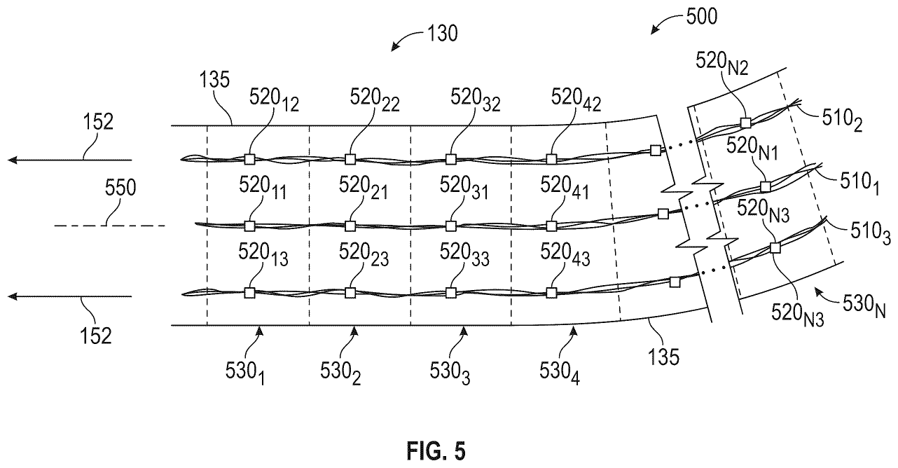

The optical receiver 184 is configured to: (i) receive returned optical signals, namely reflected light signals 150 received from optical fiber-based reflective gratings (sensors) fabricated within each core fiber of the multi-core optical fiber 135 deployed within the stylet body 290 (see FIGS. 2 and 5), and (ii) translate the reflected light signals 150 into reflection data 185, namely data in the form of electrical signals representative of the reflected light signals including wavelength shifts caused by strain. The reflected light signals 150 associated with different spectral widths may include reflected light signals 151 provided from sensors positioned in the center core fiber, e.g. center core fiber 5101, of the multi-core optical fiber 135 and reflected light signals 152 provided from sensors positioned in the periphery core fibers, e.g. periphery core fibers 5101-4, of the multi-core optical fiber 135, as described herein. Herein, the optical receiver 184 may be implemented as a photodetector, such as a positive-intrinsic-negative “PIN” photodiode, avalanche photodiode, or the like.

As shown, both the light source 182 and the optical receiver 184 are operably connected to the processor 160, which governs their operation. Also, the optical receiver 184 is operably coupled to provide the reflection data 185 to the memory 165 for storage and processing by reflection data classification logic 190. The reflection data classification logic 190 may be configured to: (i) identify which core fibers pertain to which of the received reflection data 185 and (ii) segregate the reflection data 185 provided from reflected light signals 150 pertaining to similar regions of the stylet body 290 or spectral widths into analysis groups. The reflection data for each analysis group is made available to shape sensing analytic logic 192 for analytics.

According to one embodiment of the disclosure, the shape sensing analytic logic 192 is configured to compare wavelength shifts measured by sensors deployed in each periphery core fiber at the same measurement region of the stylet body 290 (or same spectral width) to the wavelength shift at a center core fiber of the multi-core optical fiber 135 positioned along central axis and operating as a neutral axis of bending. From these analytics, the shape sensing analytic logic 192 may determine the shape the core fibers have taken in 3-D space and may further determine the current physical state of the catheter assembly 195 in 3-D space for rendering on the display 170.

According to one embodiment of the disclosure, the shape sensing analytic logic 192 may generate a rendering of the current physical state of the stylet body 290, based on heuristics or run-time analytics. For example, the shape sensing analytic logic 192 may be configured in accordance with machine-learning techniques to access a data store (library) with pre-stored data (e.g., images, etc.) pertaining to different regions of the stylet body 290 in which reflected light from core fibers have previously experienced similar or identical wavelength shifts. From the pre-stored data, the current physical state of the stylet body 290 may be rendered. Alternatively, as another example, the shape sensing analytic logic 192 may be configured to determine, during run-time, changes in the physical state of each region of the multi-core optical fiber 135 based on at least: (i) resultant wavelength shifts experienced by different core fibers within the optical fiber 135, and (ii) the relationship of these wavelength shifts generated by sensors positioned along different periphery core fibers at the same cross-sectional region of the multi-core optical fiber 135 to the wavelength shift generated by a sensor of the center core fiber at the same cross-sectional region. It is contemplated that other processes and procedures may be performed to utilize the wavelength shifts as measured by sensors along each of the core fibers within the multi-core optical fiber 135 to render appropriate changes in the physical state of the stylet body 290, especially to enable guidance of the stylet body 290, when positioned at a distal tip of the catheter assembly 195, within the vasculature of the patient and at a desired destination within the body.

The console 110 may further include electrical signal receiver logic 186, which is positioned to receive one or more electrical signals from the stylet body 290. In an embodiment, the stylet body 290 is configured to support both optical connectivity as well as electrical connectivity. The electrical signal receiver logic 186 is configured to send or receive the electrical signals or electrical energy to/from the stylet body 290 via the conductive medium 144/230.

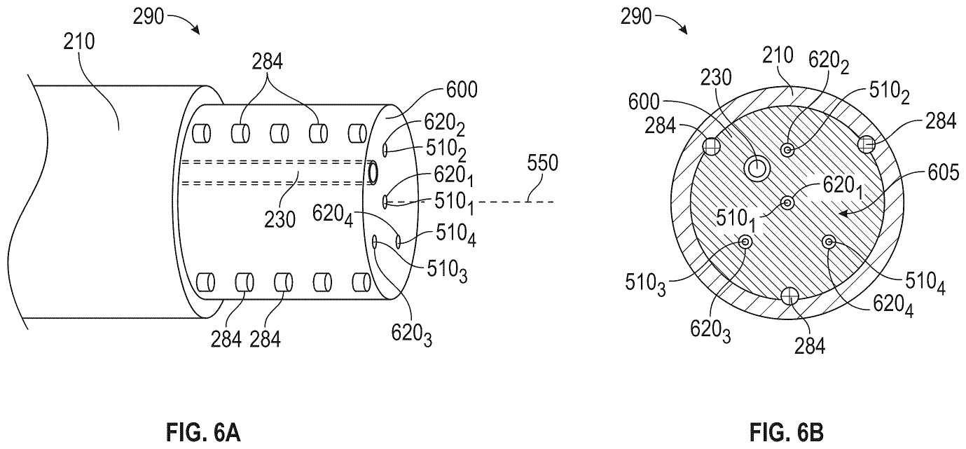

Referring now to FIG. 2, an exemplary embodiment of the stylet assembly 130. In an embodiment, as shown in FIG. 3, the stylet assembly 130 can be operably connected to a catheter assembly 195. Herein, the stylet assembly 130 features the stylet body 290, which includes an insulating layer 210 encasing a multi-core optical fiber 135 and/or conductive medium 230 (e.g. FIGS. 6A-6B). The stylet body 290 extends distally from a handle 240 while an interconnect (e.g. tether) 250 extends proximally from the handle 240 and is terminated by the console connector 132 for coupling to the interconnect 140 of the console 110 as shown in FIG. 1. The handle 240 is included with the second interconnect (e.g., tether) 250 to assist with manipulation of the stylet body 290 by the user during operation and may be configured to include activation controls 126.

As shown, the stylet body 290 and the interconnect 250 provide a pathway for outgoing optical signals produced by the light source 182 of the optical logic 180 and returning optical signals, produced by gratings within the core fibers of the multi-core optical fiber 135, for receipt by the photodetector 184 (see FIG. 1). Insulating layers associated with the stylet body 290 and the interconnect 250 may vary in density and material to control its rigidity and mechanical properties, as described herein.

Furthermore, according to one embodiment of the disclosure, the stylet assembly 130 further includes a catheter connector 270, which may be threaded for attachment to a connector of an extension leg of a catheter assembly 195 (see FIG. 3). This connectivity between the connector 270 and a connector of the extension leg connector may be used to releasably secure the stylet body 290 into a lumen of the catheter assembly 195, as shown in FIG. 3. When deployed, a distal end of the multi-core optical fiber 135 need not be substantially co-terminal with a distal tip 360 of the catheter assembly 195. As will be seen, the returned optical signals (reflected light 150) from the sensors (reflective gratings) within each core fiber included with the multi-core optical fiber 135 may be analyzed during its advancement through the patient vasculature.

Note further that, it should appreciated that the term “stylet,” as used herein, can include any one of a variety of devices configured for removable placement within a lumen of the catheter (or other portion of a medical device) to assist in placing a distal end of the catheter in a desired location within the patient's vasculature. Also, note that other connection schemes between the stylet body 290 and the console 110 can also be used without limitation.

Referring to FIG. 3, an exemplary catheter assembly 195 is provided. Herein, the catheter assembly 195 includes an elongate catheter tube 300 defining one or more lumens 310 extending between proximal and distal ends of the catheter tube 300. The catheter tube 300 is in communication with a corresponding extension leg 320 via a bifurcation hub 330. Luer connectors 340 are included on the proximal ends of the extension legs 320.

In an embodiment, the stylet assembly 130 includes the console connector 132 on its proximal end 350 to enable the stylet body 290 to operably connect with the console 110 (see FIG. 1). The interconnect 250 distally extends communications from the console 110 to the catheter connector 270, which is configured to threadably engage (or otherwise connect with) the Luer connector 340 of one of the extension legs 320 of the catheter assembly 195. The stylet body 290 extends distally from the catheter connector 270 up to a distal-end 280 of the stylet body 290. The distal-end 280 of the stylet body 290 may be substantially co-terminal with a distal tip 360 of the catheter assembly 195 within the vasculature.

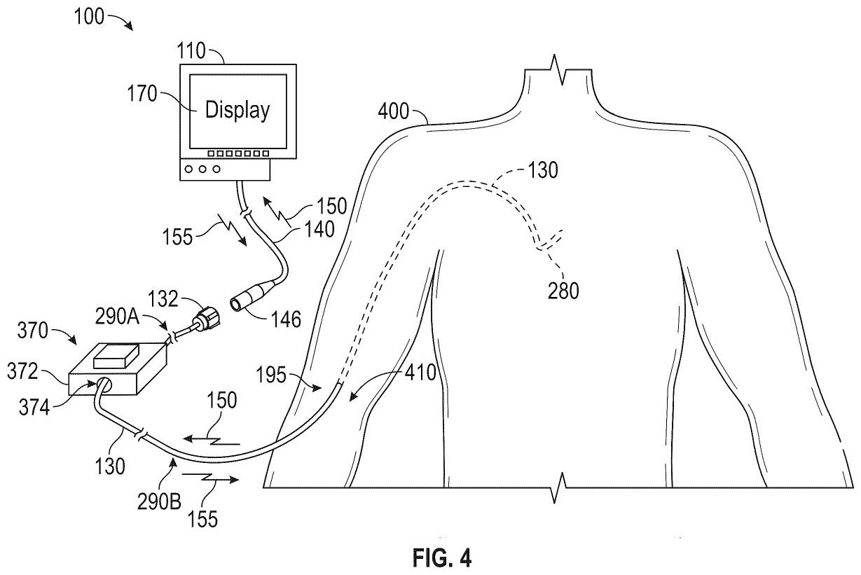

Referring now to FIG. 4, a medical device monitoring system (“system”) 100 is shown in an exemplary environment of use. In an embodiment the stylet body 290 is being inserted into a vasculature of a patient 400 through a skin insertion site 410. In an embodiment, the stylet body 290 is advanced through the vasculature to a target location within the patient vasculature. In one embodiment, the target location for the stylet distal tip 280 is proximate the patient's heart, such as in the lower one-third (⅓^{rd}) portion of the Superior Vena Cava (“SVC”) for this embodiment. Of course, other target locations within the body of the patient are also contemplated.

During advancement of the stylet assembly 130, the stylet body 290 receives broadband light 155 from the console 110 via interconnect 140, which includes the connector 146 for coupling to the console connector 132 for the stylet assembly 130. The reflected light 150 from sensors (reflective gratings) within each core fiber of the multi-core optical fiber 135 are returned from the stylet body 290 over the interconnect 140 for processing by the console 110. The physical state of the stylet body 290 may be ascertained based on analytics of the wavelength shifts of the reflected light 150. For example, the strain caused through bending of the stylet body 290, and hence angular modification of each core fiber, causes different degrees of deformation. The different degrees of deformation alters the shape of the sensors (reflective grating) positioned on the core fiber, which may cause variations (shifts) in the wavelength of the reflected light from the sensors positioned on each core fiber within the multi-core optical fiber 135, as shown in FIG. 5. From this wavelength shifting, the shape sensing analytic logic 192 within the console 110 (see FIG. 1) may determine the positional information of the stylet body 290 (e.g., shape, location, orientation, etc.).

Referring to FIG. 5, an exemplary embodiment of a right-sided, longitudinal view of a section 500 of the multi-core optical fiber 135 included within the stylet body 290 is shown. The multi-core optical fiber section 500 depicts certain core fibers 510₁-510_{M} (M≥2, M=4 as shown) along with the spatial relationship between sensors (e.g., reflective gratings) 520₁₁-520_{NM} (N≥2; M≥2) present within the core fibers 510₁-510_{M}, respectively. As shown, the section 500 is subdivided into a plurality of cross-sectional regions 530₁-530_{N}, where each cross-sectional region 530₁-530_{N} corresponds to reflective gratings 520₁₁-520₁₄ . . . 520_{N1}-520_{N4}. Some or all of the cross-sectional regions 530₁ . . . 530_{N} may be static (e.g., prescribed length) or may be dynamic (e.g., vary in size among the regions 530₁ . . . 530_{N}). A first core fiber 510₁ is positioned substantially along a center (neutral) axis 550 while core fiber 510₂ may be oriented within the cladding of the multi-core optical fiber 135, from a cross-sectional, front-facing perspective, to be position on “top” the first core fiber 510₁. In this deployment, the core fibers 510₃ and 510₄ may be positioned “bottom left” and “bottom right” of the first core fiber 510₁. (See FIG. 6B).

Referencing the first core fiber 510₁ as an illustrative example, when the stylet body 290 is operative, each of the reflective gratings 520₁-520_{N} reflect light for a different spectral width. As shown, each of the gratings 520₁ᵢ-520_{Ni} (1≤i≤M) is associated with a different, specific spectral width, which would be represented by different center frequencies of ƒ₁ . . . ƒ_{N}, where neighboring spectral widths reflected by neighboring gratings are non-overlapping according to one embodiment of the disclosure.

Herein, positioned in different core fibers 510₂-510₃ but along at the same cross-sectional regions 530-530_{N} of the multi-core optical fiber 135, the gratings 520₁₂-520_{N2} and 520₁₃-520_{N3} are configured to reflect incoming light at same (or substantially similar) center frequency. As a result, the reflected light returns information that allows for a determination of the physical state of the optical fiber 135 (and the stylet body 290) based on wavelength shifts measured from the returned, reflected light. In particular, strain (e.g., compression or tension) applied to the multi-core optical fiber 135 (e.g., at least core fibers 510₂-510₃) results in wavelength shifts associated with the returned, reflected light. Based on different locations, the core fibers 510₁-510₄ experience different types and degree of strain based on angular path changes as the stylet body 290 advances in the patient.

For example, with respect to the multi-core optical fiber section 500 of FIG. 5, in response to angular (e.g., radial) movement of the stylet body 290 is in the left-veering direction, the second core fiber 510₂ of the multi-core optical fiber 135 with the shortest radius during movement (e.g., core fiber closest to a direction of angular change) would exhibit compression (e.g., forces to shorten length). At the same time, the third core fiber 510₃ with the longest radius during movement (e.g., core fiber furthest from the direction of angular change) would exhibit tension (e.g., forces to increase length). As these forces are different and unequal, the reflected light from reflective gratings 520_{N2} and 520_{N3} associated with the core fiber 510₂ and 510₃ will exhibit different changes in wavelength. The differences in wavelength shift of the reflected light signals 152 can be used to extrapolate the physical configuration of the stylet body 290 by determining the degrees of wavelength change caused by compression/tension for each of the periphery fibers (e.g., the second core fiber 510₂, the third core fiber 510₃, and the fourth core fiber 510₄) in comparison to the wavelength of the reference core fiber (e.g., first core fiber 510₁) located along the neutral axis 550 of the multi-core optical fiber 135. These degrees of wavelength change may be used to extrapolate the physical state of the stylet body 290.

Referring now to FIGS. 6A-6B, an exemplary embodiment of a multimodal stylet body 290 of FIG. 1 supporting one or both of an optical and electrical signaling is shown. Herein, the stylet body 290 features a centrally located multi-core optical fiber 135, which includes a cladding 600 and a plurality of core fibers 510₁-510_{M} (M≥2; M=4) residing within a corresponding plurality of lumens 620₁-620_{M}. While the multi-core optical fiber 135 is illustrated within four (4) core fibers 510₁-510₄, a greater number of core fibers 510₁-510_{M} (M>4) may be deployed to provide a more detailed three-dimensional sensing of the positional information of the multi-core optical fiber 135 and the stylet body 290 deploying the optical fiber 135, a greater number of core fibers 510₁-510_{M} (M>4) may be deployed. In an embodiment, the optical fiber 135 can include an outer insulating layer 210 disposed on an outer surface thereof. In an embodiment, the stylet body 290 can include a conductive medium 230 extending therethrough to provide an electrically conductive pathway therethrough.

Further details, examples and embodiments of fiber-optic enabled strain sensor (FOSS) systems can be found in U.S. 2018/0289927, U.S. 2021/0045814, U.S. 2021/0156676, U.S. 2021/0154440, U.S. 2021/0275257, U.S. 2021/0268229, U.S. 2021/0271035, U.S. 2021/0402144, U.S. 2021/0401509, U.S. 2022/0011192, and U.S. 2022/0034733, each of which are incorporated by reference in their entirety.

In further reference to FIG. 4, in an embodiment the stylet body 290 can be configured to be separated at one or more positions along a longitudinal length thereof. In an embodiment the stylet body 290 can be configured to be subsequently reconnected at the position of the one or more positions of separation. In an embodiment, the stylet body 290 includes a continuous cross-sectional structure, as shown in FIGS. 6A-6B and is configured to be cut at any position along a longitudinal length thereof. In an embodiment, the clinician can cut the stylet body 290 using a scissors, scalpel, knife, pliers, or similar cutting instrument.

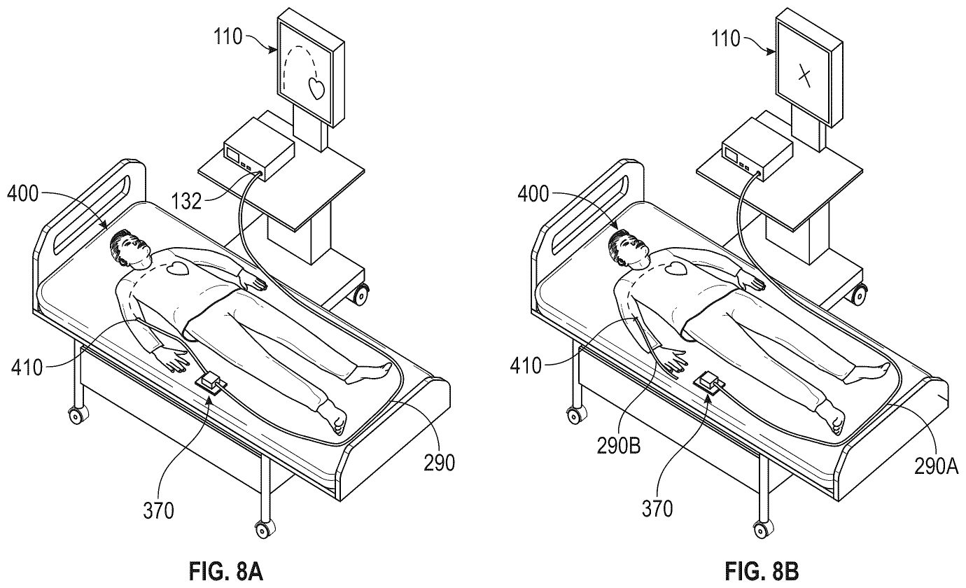

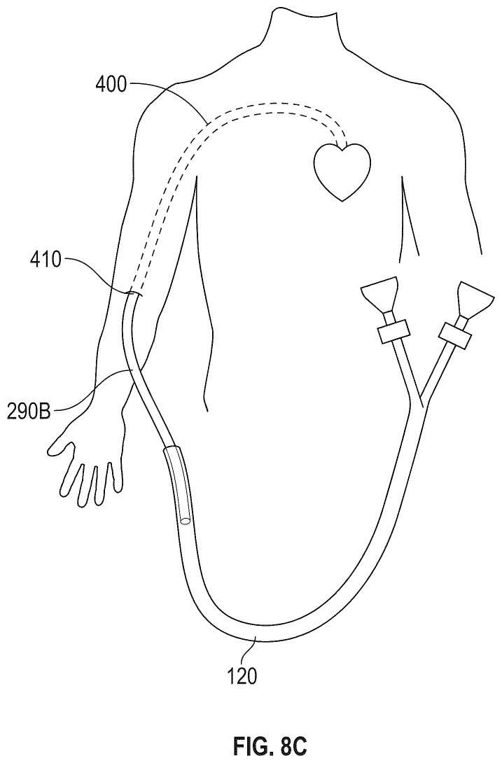

In an exemplary method of use the stylet distal tip 280 can be advanced to a target location as described herein. The clinician can then cut a proximal portion of the stylet body 290 to separate distal portion 290B of the stylet body 290 from the proximal portion 290A coupled to the console connector 132. A catheter assembly 120 can then be advanced over the distal portion 290B of the stylet body 290 until a distal tip 360 of the catheter assembly 120 is disposed at the target location within the patient 400. (FIG. 8C) Once the catheter assembly 120 is placed, the distal portion 290B of the stylet body 290 can be withdrawn proximally from a lumen of the catheter assembly 120.

In an embodiment, the stylet body 290 includes one or more break lines, such as a score line, groove, perforation, or similar line of weakness extending laterally through the stylet body 290 and configured to facilitate separation of the stylet body 290 a one or more predetermined positions along a longitudinal length thereof. In an exemplary method of use, a clinician can cut or bend the stylet body 290 adjacent a break line of the one or more break lines to rupture the stylet body 290 at the break line and separate a distal portion 290B of the stylet body 290 from a proximal portion. The catheter assembly 120 can be advanced over the distal portion 290B of the stylet body 290, as described herein.

In an embodiment, the stylet body 290 can be formed of one or more stylet body sections, each having the same cross-sectional structure as shown in FIGS. 6A-6B. A proximal end of a first stylet body section can be coupled to a distal end of a second stylet body section using one or more of a latching mechanism, friction fit collar, press fit collar, snap fit collar, threaded engagement, magnetic engagement, combinations thereof, or the like. In an exemplary method of use, once the stylet distal tip 280 is advanced to a target location, one or more proximal stylet body sections can be separated from one or more distal stylet body sections but uncoupling the latching mechanism, collar, threaded engagement, etc. and a catheter assembly 120 advanced of the one or more distal stylet body sections as described herein.

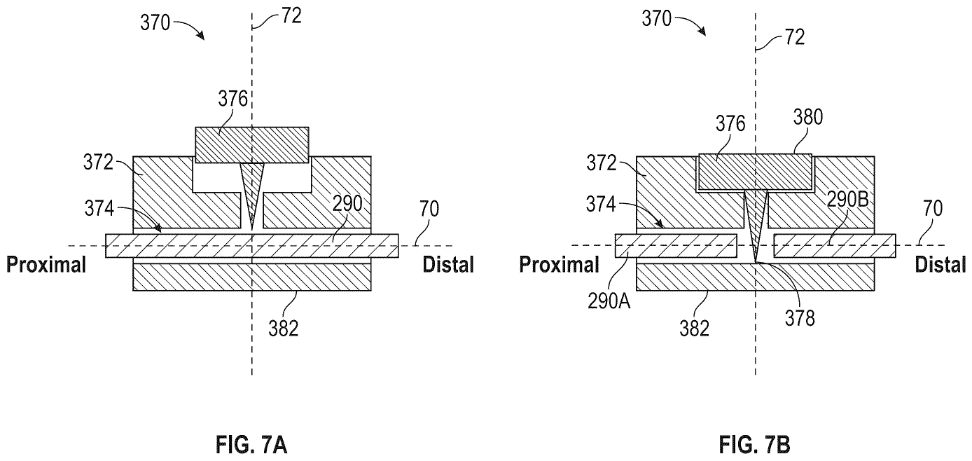

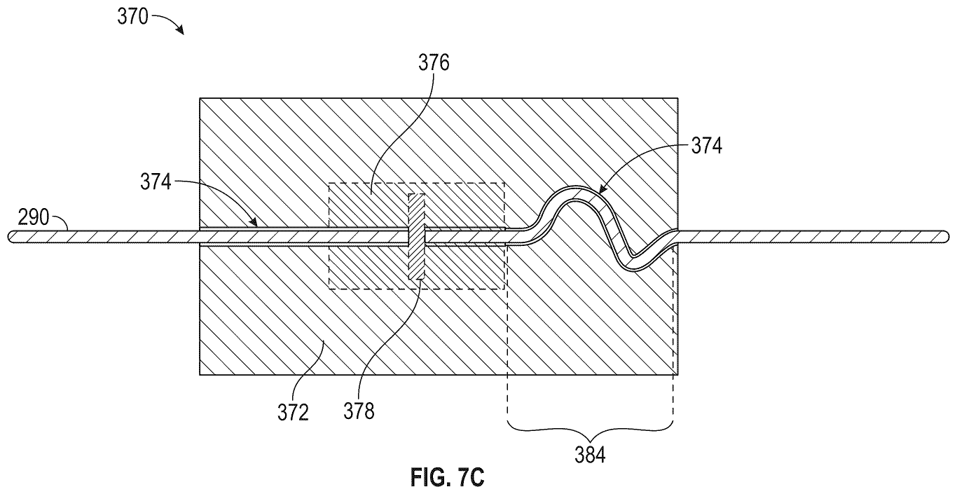

In an embodiment, the system 100 further includes severing device 370. FIGS. 7A-7B show longitudinal cross-sectional views of the severing device 370 with a portion of the stylet body 290 extending therethrough. The severing device 370 includes a body 372 having a base 382 on a lower side, and having a channel 374 extending through the body substantially parallel to the base 382, although other angles are contemplated. The severing device 370 further includes an actuator 376 slidably engaged with the body 371 and having a cutting edge 378 and an actuator surface 380. The channel 374 defines a circular cross-sectional shape having an inner diameter equal to, or slightly larger than an outer diameter of the stylet body 290. As such, the stylet body 290 can slidably engage channel 374.

The actuator 376 is configured to transition between a retracted position (FIG. 7A) and an extended position (FIG. 7B) relative to the body 372 and channel 374, and along an axis 72 extending perpendicular to a central axis 70 of the channel 374. The actuator 376 is biased towards the retracted position, and in use, a user can urge the actuator 376 to the extended position by applying a force to the actuator surface 380 and urging the cutting edge 378 through the channel 374, perpendicular to the longitudinal axis 70 of the stylet body 290, severing the stylet body 290.

The base 382 of the body 372 can support the severing device 370 and stabilize the severing device 370 when placed on a surface 402, e.g. operating table, bed, work surface, or the like. Optionally, the base 382 can include an adhesive, magnet, hoop and loop fastener, or similar means of releasably securing the base 382 to the surface. The actuator surface 380 extends from a top side of the body 372. Advantageously, the severing device 370 is configured to sever the stylet body 290 perpendicular to the longitudinal axis 70. The cut ends of the stylet body 290 provide a perpendicular surface relative to the axis of the optical fiber 135, allowing the ends to be reconnected flush with each other, mitigating reflection or refraction of light at the interface between the cut ends of the optical fiber 135. This allows for continued optical transmission of light across the cut ends of the optical fiber 135 while mitigating distortion or disruption of the light signals.

In an exemplary method of use, as shown in FIGS. 8A-8B, a system 100 is provided including a stylet body 290 and a severing device 370, as described herein. A patient 400 is placed on a surface 402, e.g. an operating table, with the console 110 adjacent. The stylet body 290 extends from the console connector 132, through the severing device channel 374, through the insertion site 410 and into the vasculature of the patient 400. The base 382 of the severing device 370 can be secured adjacent the patient 400. The stylet body 290 is slidably engaged with channel 374 to allow the stylet body 290 to be advanced therethrough and into the insertion site 410 with little or no resistance. Further, the channel 374 maintains the longitudinal axis 70 of the stylet body 290 perpendicular to the axis 72 of the cutting edge 378. Advantageously, the channel ensures the severing device 370 severs the stylet body 290 perpendicular to the longitudinal axis 70. This allows the cut ends to be reconnected and mitigate reflection or refraction of the light signals at the cut interface.

In an embodiment, the severing device 370 is disposed within the sterile field and adjacent the patient 400 to allow a clinician to easily access and actuate the actuator 376. In an embodiment, the severing device 370 is disposed adjacent the patient 400 and outside of the sterile field. Advantageously, the actuator 376 is sized large enough to allow the clinician to easily access and actuate through the actuator 376 through the sterile barrier. Optionally, the severing device 370 can include one or more lights or palpation features to facilitate a clinician to locate the severing device 370 and actuator surface 380 through a sterile barrier. Advantageously, the severing device 370 is easily actuated, even when the clinician has reduced dexterity. For example, if the clinician's hands are engaged with operating or positioning the stylet assembly 130, the severing device 370 can still be easily actuated. Further, the clinician does not need to take time in aligning a cutting implement at a precise perpendicular angle since the severing device 370 aligns the stylet body 290 relative to the cutting edge 378.

As shown in FIGS. 8A-8B, once the distal tip 280 of the stylet body 290 is advanced to the target location, the clinician can actuate the actuator surface 380 to sever the stylet body 290 and separate a distal portion 290B of the stylet body 290 from a proximal portion 290A, still coupled to the console 110. The severing device 370 can ensure a clean, perpendicular cut through the longitudinal axis 70 of the stylet body 290. As shown in FIG. 8C, a catheter assembly 120 can then be advanced over the distal portion 290B of the stylet body 290 until the catheter distal tip 360 is disposed at the target location. In an embodiment, the distal portion 290B of the stylet body 290 can then be withdrawn from the lumen of the catheter assembly 120.

In an embodiment, a proximal end of the stylet distal portion 290B can be coupled with the distal end of the stylet proximal portion 290A. The severing device 370 ensures a precise, perpendicular cut to facilitate reconnection of the optical fiber 135 and/or conductive medium 230 between the proximal end of the stylet distal portion 290B and the distal end of the stylet proximal portion 290A. Advantageously, the clinician can reconnect the stylet body 290 to confirm the catheter distal tip 360 is at the target location before removing the stylet body 290 from the catheter assembly 120.

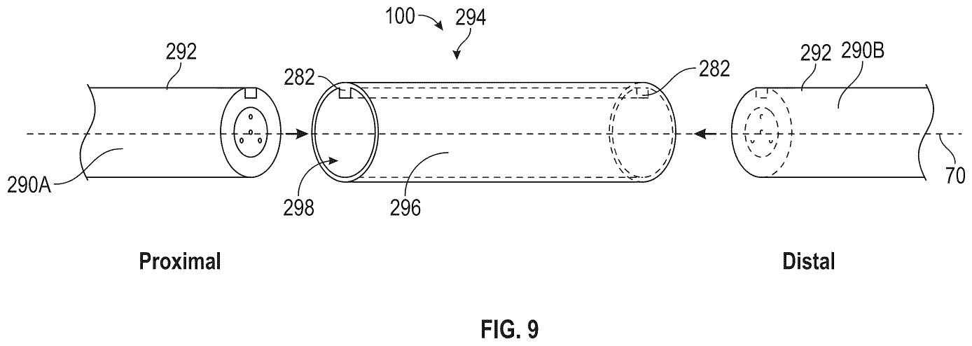

FIG. 9 shows close up detail of an exemplary connection point between the proximal end of the stylet distal portion 290B and the distal end of the stylet proximal portion 290A. In an embodiment, the stylet body 290 includes a keying feature 292 extending longitudinally along a length of the stylet body 290. Exemplary keying features include a groove, facet, ridge, combinations thereof, or the like.

In an embodiment, a connector 294 is provided having a body 296 extending between a first end and a second end and defining a lumen 298 extending therebetween. An inner diameter of the lumen 298 having a diameter equal to, or slightly larger than, an outer diameter of the stylet body 290 and configured to slidably engage the stylet body 290. The connector 294 further includes an alignment feature 282 extending longitudinally along an inner surface of the connector lumen 298 and configured to engage the keying feature 292 of the stylet body 290 to orient the proximal end of the stylet distal portion 290B with the distal end of the stylet proximal portion 290A about the longitudinal axis 70.

For example, as shown, the keying feature 292 includes a groove extending longitudinally along a top side of the stylet body 290. The keying feature 292 is sized and shaped to receive the alignment feature 282 only when the distal end of the stylet proximal portion 290A is oriented with the keying feature 292 on the top side and prevents the stylet proximal portion 290A from engaging the connector 294 if oriented in any other position. Similarly, the keying feature 292 of the proximal end of the stylet distal portion 290B is configured to engage the connector 294 only when the keying feature 292 is oriented on the top side. The stylet proximal portion 290A and the stylet distal portion 290B are then advanced into the connector lumen 298 until the proximal end of the stylet distal portion 290B and the distal end of the stylet proximal portion 290A engage, aligning the optical fiber 135 and conductive medium 230 and reestablishing connection therebetween.

Advantageously, the proximal end of the stylet distal portion 290B and the distal end of the stylet proximal portion 290A are cut perpendicular to allow the ends to engage over an entire surface allowing the optical fiber 135/conductive element 230 to re-establish connection across the cut end interface. As will be appreciated, if one or both of the cut ends are at an angle, ends may not engage over an entire surface, leaving a gap therebetween and disrupting optical/electrical communication across the interface.

As will be appreciated, the keying feature 292 and the alignment feature 282 can be a ridge or groove, a facet engaging a facet or any suitable cross-sectional shape to ensure the stylet proximal portion 290A and the stylet distal portion 290B are oriented at the same angle of rotation about the longitudinal axis 70. In an embodiment, the keying feature 292 and the alignment feature 282 can be magnetic, i.e. the keying feature 292 is a first magnetic element and the alignment feature 282 is a second magnetic element configured to be attracted to the first magnetic element and orientate the proximal end of the stylet distal portion 290B and the distal end of the stylet proximal portion 290A, as described herein. As used herein, a magnetic element includes a permanent magnet (e.g. Neodymium magnets, ferrite magnets, or the like), electro-magnets (e.g. a material conductive to an electrical current), a magnetizable material (e.g. a ferrous material exposed to a magnetic field to become magnetic), or combinations thereof.

In an embodiment, the connector 294 engages the proximal end of the stylet distal portion 290B and the distal end of the stylet proximal portion 290A in a friction fit engagement, press fit engagement, snap fit engagement, threaded engagement, bayonet engagement, latching mechanism, adhesive, bonding, welding, ultrasonic welding, RF energy welding, or similar suitable means.

In an embodiment, once connected with the connector 294, the proximal end of the stylet distal portion 290B and the distal end of the stylet proximal portion 290A can be subsequently disconnected from the connector 294. For example, the threaded or bayonet connection can be rotated to unscrew the respective proximal and distal ends from the connector 294. Further, for frictional or press fit engagements the proximal and distal ends can be urged out of the connector 294 with sufficient force to overcome the force of the frictional or press fit engagements. For latching or snap fit engagements, the mechanism can be actuated to release or uncouple the respective proximal and distal ends from the connector 294. Adhesive connections can be dissolved using solvents, or cut using a knife, scissors, or severing device, as described herein. These and other means of releasably disconnecting the connection means are also contemplated to fall within the scope of the present invention.

In an embodiment, as shown in FIGS. 6A-6B, the stylet body 290 can further include one or more magnetic elements 284 disposed about the central longitudinal axis and extending longitudinally through the stylet body 290 either continuously, or discontinuously. As shown in FIG. 6B, three magnetic elements 284 are disposed radially evenly about a central longitudinal axis, although other numbers and configurations of magnetic elements 284 are contemplated. As shown in FIG. 6A, the magnetic elements 284 extend discontinuously through the longitudinal length of the stylet body 290. Advantageously, the magnetic elements 284 are configured to orient and couple the proximal end of the stylet distal portion 290B and the distal end of the stylet proximal portion 290A together, optionally with or without the use of the connector 294.

As described herein, the stylet body 290 can be severed perpendicular to the longitudinal axis, as such a first magnetic element 284 is disposed at the proximal end of the stylet distal portion 290B and a second magnetic element 284 is disposed at the distal end of the stylet proximal portion 290A. A clinician can reconnect the proximal end of the stylet distal portion 290B and the distal end of the stylet proximal portion 290A by placing the respective proximal and distal ends together, allowing the magnetic elements 284 to attract each other pulling the respective proximal and distal ends together and reconnecting the optical fiber 135 and conductive medium 230.

In an embodiment, as shown in FIG. 6B a polar orientation of a first magnetic element 284, e.g. a bottom magnetic element, can be different from a polar orientation of a second magnetic element 284, e.g. a top left or top right magnetic element. Accordingly, if the proximal end of the stylet distal portion 290B and the distal end of the stylet proximal portion 290A are misaligned about the longitudinal axis 70, the magnetic elements 284 will also be misaligned and either fail to attract, or even repel, the proximal end of the stylet distal portion 290B and the distal end of the stylet proximal portion 290A. Only when the proximal end of the stylet distal portion 290B and the distal end of the stylet proximal portion 290A are correctly aligned about the longitudinal axis then the polar orientation of the magnetic elements 284 attract the proximal end of the stylet distal portion 290B and the distal end of the stylet proximal portion 290A together.

FIG. 7C shows a plan view of the severing device 370 showing a channel 374 extending therethrough and a position of the actuator 376 and cutting edge 378 shown in outline for ease of illustration. In an embodiment, the channel 374 of the severing device 370 can include a shape sensing fiduciary marker 384. As used herein, a fiduciary marker 384 is a landmark used by the system 100 to establish a set point in three-dimensional space. From this fiduciary marker, the relative positions of other points in three-dimensional space, determined by the system 100, can be set. As described herein, the console 110 is configured to detect a shape of the optical fiber 135 by detecting a direction and angle of flexion relative to a central longitudinal axis 70. As shown in FIG. 7C, a portion of the channel 374 can define a specific shape having a predetermined direction and angle of flexion from a central longitudinal axis to provide a fiduciary marker 384.

As the stylet body 290, including the optical fiber 135, is advanced through the fiduciary marker 384 of the channel 374, the console 110 can detect the predetermined shape of the fiduciary marker 384. The system 100 can then determine the position of the severing device 370 relative to the stylet body 290. Further, as the stylet body 290 is advanced through the channel 374, the position of the stylet distal tip 280 can be calculated relative to the fiduciary marker 384. As shown the fiduciary marker 384 of the channel 374 is shown as a sigmoid curve, however other shapes and angles are also contemplated to fall within the scope of the present invention. Advantageously, the severing device need not be communicatively coupled with the console 110 since the fiduciary marker of the channel 374 can be detected by the stylet body 290. In an embodiment, the severing device 370 is communicatively coupled to the console 110 and includes one or more sensors configured to record the presence of the stylet body 290 in the channel 374, actuation of the actuator 376, etc. The number of these events and associated date/time stamps can be recorded by the console 110.

In an embodiment, the console 110 is configured to detect a position of the cut of the stylet body 290. For example, the console 110 is configured to detect a longitudinal distance between a distal tip 280 of the stylet body 290 and the fiduciary marker 384 of the channel 374. When the clinician actuates the severing device 370 a distance between the distal end of the stylet proximal portion 290A and the fiduciary marker 384. Accordingly, the console 110 can also record a date/time of when the stylet body 290 can severed, a longitudinal distance, an insertion length, and shape of the stylet distal portion 290B and/or the stylet proximal portion 290A at the time of severing, and other diagnostic information such as kinking, looping, blockages, and advancement attempts.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A catheter placement system for placing a catheter within a vasculature of a patient, comprising:
a stylet having a body extending along a longitudinal axis between a console connector at a proximal end and a distal tip, the stylet body including an optical fiber extending therethrough, the stylet body severable along an axis extending perpendicular to the longitudinal axis to separate a distal portion of the body from a proximal portion of the body and configured to be reconnected to communicatively couple the optical fiber of the distal portion with the optical fiber of the proximal portion; and
a console having an optical logic and providing broadband incidence light to the optical fiber and configured to receive reflected light signals from the optical fiber, the optical logic configured to determine positional information about the stylet body.

2. The catheter placement system according to claim 1, wherein the catheter is configured to slidably engage the severed proximal end of the stylet body distal portion to advance a catheter distal tip to a target location within the vasculature of the patient.

3. The catheter placement system according to claim 1, further including a severing device having a body defining a channel, a portion of the stylet body extending through the channel and an actuator including a cutting edge configured to cut the stylet body perpendicular to the longitudinal axis of the stylet body.

4. The catheter placement system according to claim 1, wherein the stylet body includes a keying feature extending along a longitudinal length of the body and configured to orient the stylet distal portion about the longitudinal axis relative to the stylet proximal portion.

5. The catheter placement system according to claim 4, further including a connector having a body defining a lumen and including an aligning feature extending along an inner surface of the connector lumen, the aligning feature configured to engage a first keying feature of the stylet distal portion and a second keying feature of the stylet proximal portion to align an optical fiber end of the stylet distal portion with an optical fiber end of the stylet proximal portion.

6. The catheter placement system according to claim 5, wherein the connector engages one or both of the stylet distal portion and the stylet proximal portion in one of a friction fit engagement, a press fit engagement, a snap fit engagement, a threaded engagement, a bayonet engagement, a latching engagement, an adhesive, a bonding, a welding, an ultrasonic welding, or an RF energy welding.

7. The catheter placement system according to claim 3, wherein the channel of the severing device includes a fiduciary marker having a predetermined non-linear path to a portion of the channel.

8. The catheter placement system according to claim 1, wherein the stylet body includes a plurality of magnetic elements extending longitudinally through the stylet body and configured to releasably attach a proximal end of the stylet distal portion to a distal end of a stylet proximal portion.

9. A method of placing a catheter, comprising:
advancing a distal end of a body of a stylet through an insertion site to a target location;
sending broadband incident light from a console to an optical fiber of the stylet body;
receiving reflected light to the console to determine positional information of the stylet body;
confirming a distal end of the stylet body is at the target location;
severing a distal portion of the stylet body from a proximal portion of the stylet body;
advancing a catheter over the stylet distal portion subsequent to severing the distal portion of the stylet body; and
removing the stylet distal portion from a lumen of the catheter.

10. The method according to claim 9, further including coupling the proximal end of the distal portion of the stylet body with a distal end of the proximal portion of the stylet body subsequent to the step of advancing the catheter over the stylet distal portion and receiving reflected light from the distal portion to determine positional information of the distal portion.

11. The method according to claim 10, wherein coupling the proximal end of the distal portion of the stylet body with the distal end of the proximal portion of the stylet body includes aligning a keying feature of the stylet body with an alignment feature of a connector, the keying feature extending along a longitudinal length of the body.

12. The method according to claim 11, wherein the connector has a connector body defining a lumen, the alignment feature extending longitudinally along an inner surface of the lumen to align an optical fiber end of the stylet distal portion with an optical fiber end of the stylet proximal portion.

13. The method according to claim 12, wherein the connector engages one or both of the stylet distal portion and the stylet proximal portion in one of a friction fit engagement, a press fit engagement, a snap fit engagement, a threaded engagement, a bayonet engagement, a latching engagement, an adhesive, a bonding, a welding, an ultrasonic welding, or an RF energy welding.

14. The method according to claim 9, wherein severing the distal portion from the proximal portion further includes actuating a severing device having a body defining a channel, a portion of the stylet body extending through the channel and an actuator including a cutting edge transitionable along an axis perpendicular to a longitudinal axis of the stylet body.

15. The method according to claim 14, wherein the channel of the severing device includes a fiduciary marker having a predetermined non-linear path to a portion of the channel.

16. The method according to claim 9, wherein the stylet body includes a plurality of magnetic elements extending longitudinally through the stylet body and configured to releasably attach a proximal end of the stylet distal portion to a distal end of a stylet proximal portion.
